(19) United States
(12) Patent Application Publication
(10) Pub. No.: US 2018/0036465 A1
(43) Pub. Date: Feb. 8, 2018

(54) EVACUATING FLUID SURROUNDING DEVICES IMPLANTED IN BODY

(71) Applicant: HeartWare, Inc., Miami Lakes, FL (US)

(72) Inventor: John RUDSER, Miami, FL (US)

(21) Appl. No.: 15/670,693

(22) Filed: Aug. 7, 2017

Related U.S. Application Data
(60) Provisional application No. 62/372,061, filed on Aug. 8, 2016.

(51) Int. Cl. A61M 1/12; A61M 1/10

(52) U.S. Cl. CPC A61M 1/12; A61M 1/1012; A61M 1/1086; A61B 2017/00243

(57) ABSTRACT

A method and kit for placing an implant in a patient. The method includes placing an implant consisting of at least one from the group consisting of an electronic device and an electromechanical device into a tissue cavity of the patient and evacuating fluid from the cavity and urging tissue surrounding the cavity to abut the implant.

### CROSS-REFERENCE TO RELATED APPLICATION

This application is related to and claims priority to U.S. Provisional Patent Application Ser. No. 62/372061, filed Aug. 08, 2016, entitled EVACUATING FLUID SURROUNDING DEVICES IMPLANTED IN BODY, the entirety of which is incorporated herein by reference.

### STATEMENT REGARDING FEDERALLY SPONSORED RESEARCH OR DEVELOPMENT

n/a

### TECHNICAL FIELD

The present disclosure relates to kits and methods for implanting objects in the bodies of patients.

### BACKGROUND

Certain surgeries involve implantation of an object in a cavity within the body of the patient. An object introduced within the body in a medical procedure and intended to remain in the body after completion of the procedure is referred to herein as an “implant.” For example, implantation of certain mechanical circulatory support devices (“MCSDs”) used to assist the pumping action of the heart involve implantation of a battery in a surgically-created cavity within the body. Such a battery or other implantable electronic device can provide power to the MCSD while the external power supply to the MCSD is temporarily interrupted. In some cases, the tissue surrounding the implant does not heal quickly or properly. This may leave the implant free to move within the cavity as the patient changes position, and may also lead to inflammation or even necrosis of tissue surrounding the cavity. This problem is particularly acute in cases where the implant is a relatively heavy object such as battery for an MCSD. Further improvement would be desirable.

### SUMMARY

The present invention advantageously provides a method and kit for placing an implant in a patient. The method includes placing an implant consisting of at least one from the group consisting of an electronic device and an electromechanical device into a tissue cavity of the patient and evacuating fluid from the cavity and urging tissue surrounding the cavity to abut the implant.

In another aspect of this embodiment, the cavity is surgically created.

In another aspect of this embodiment, placing the implant into the cavity includes placing the implant through the surgically created opening in the tissue of the patient.

In another aspect of this embodiment, the method includes accessing the cavity through the opening to evacuate fluid in the cavity.

In another aspect of this embodiment, evacuating fluid from the cavity includes lowering the pressure within the cavity to a sub-atmospheric pressure.

In another aspect of this embodiment, the method includes partially closing the opening prior to evacuating fluid from the cavity.

In another aspect of this embodiment, the method includes interrupting evacuating fluid by at least a partial closing of the opening in the tissue prior to completing the evacuation of the fluid.

In another aspect of this embodiment, evacuating fluid is performed for at least 12 hours and a sub-atmospheric pressure is maintained within the cavity for at least 12 hours.

In another aspect of this embodiment, evacuating fluid is performed until at least a portion of the tissue abuts the implant.

In another aspect of this embodiment, evacuating fluid is performed until at least one from the group consisting of the tissue surrounding the cavity at least partially heals and the tissue surrounding the cavity secures the tissue with the implant.

In another aspect of this embodiment, the method includes closing the opening in the tissue and subsequently reopening the closed opening and further comprising at least one from the group consisting of evacuating any fluid not previously removed and evacuating any fluid that has accumulated after closing the opening.

In another aspect of this embodiment, the implant includes a tissue ingrowth promoter on a surface of the implant, and wherein the tissue ingrowth promoter includes a surface with openings.

In another aspect of this embodiment, evacuating fluid includes holding tissue of the cavity in abutment with the implant for a duration sufficient for tissue ingrowth into the implant to begin.

In another aspect of this embodiment, the implant is one from the group consisting of a battery, pump, controller, wireless power receiver, transcutaneous connector, and any combination thereof.

In another aspect of this embodiment, evacuating fluid includes inserting a transcutaneous tube into an opening in the tissue of the patient and evacuating fluid through the transcutaneous tube.

In another embodiment, the kid includes an implant including at least one from the group consisting of an electronic device and electromechanical device sized be disposed in a surgically cavity of a patient. A tube is sized to be received within the cavity. A pump is connectable to the tube, the pump being configured generate negative pressure that causes suction in the tube.

In another aspect of this embodiment, the implant is one of a battery, implantable pump, controller, wireless power receiver, transcutaneous connector, or any combination thereof.

In another aspect of this embodiment, the tube includes a suction catheter with a needle configured to penetrate tissue of the patient.

In another aspect of this embodiment, the kit includes a collection canister, the collection canister being connectable with the tube and configured house fluids suctioned through the tube.

In yet another embodiment, a method of placing an implant in a patient includes surgically creating a tissue cavity in the patient. An implant consisting of a battery is placed into the tissue cavity. The tissue cavity is partially closed. A transcutaneous tube is inserted into the partially closed tissue cavity. Fluid is evacuated from the cavity through the transcutaneous tube and tissue surrounding the cavity is urged to abut the implant. The pressure is lowered within the cavity to a sub-atmospheric pressure.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a diagrammatic perspective view illustrating a kit in accordance with one embodiment of the invention;

FIG. 2 is a diagrammatic perspective view illustrating one step of a method according to one embodiment of the invention, employing the kit of FIG. 1;

FIG. 3A is a diagrammatic perspective view depicting the area indicated in FIG. 2 on an enlarged scale;

FIG. 3B is a diagrammatic, fragmentary sectional view along line 3B-3B in FIG. 3A;

FIGS. 4A and 4B are views similar to FIGS. 3A and 3B but illustrating another step in the method; and

FIG. 5 is a view similar to FIGS. 3A and 4A but depicting a later step in the method.

### DETAILED DESCRIPTION

The various devices, kits and associated methods of use of the present invention are intended for removal and otherwise accelerating the removal of fluid cavities within a patient, particularly cavities adjacent to and surrounding devices implanted under a patient's tissue. The term “implant” as used herein refers to any device or other structure intentionally left within the body following surgery.

The term “cavity” as used throughout is intended to refer generally to spaces within the human body. A “cavity” can include either a natural cavity or a surgically created cavity, where a surgically created cavity, or surgical cavity, is one that is either created or develops in conjunction with surgery. Of course, where the term “natural cavity” or “surgical cavity” is used, the meaning corresponding to those terms applies.

The term “atmospheric pressure” as used throughout is intended to refer generally to the pressure exerted by the weight of air in the atmosphere of earth. This pressure varies based on local climate conditions, but is generally within several kilopascals of standard atmospheric pressure, 101.3 kPa. The terms “sub-atmospheric pressure” or “negative pressure” as used throughout are intended to refer generally to a pressure in a defined space that is lower than the ambient pressure immediately outside of that space, such as the pressure in a cavity relative to the pressure in the tissue surrounding the cavity.

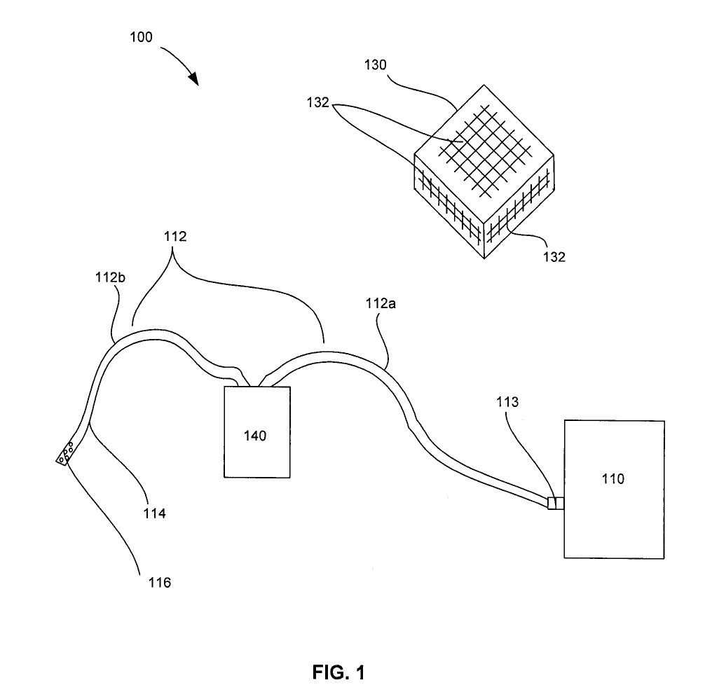

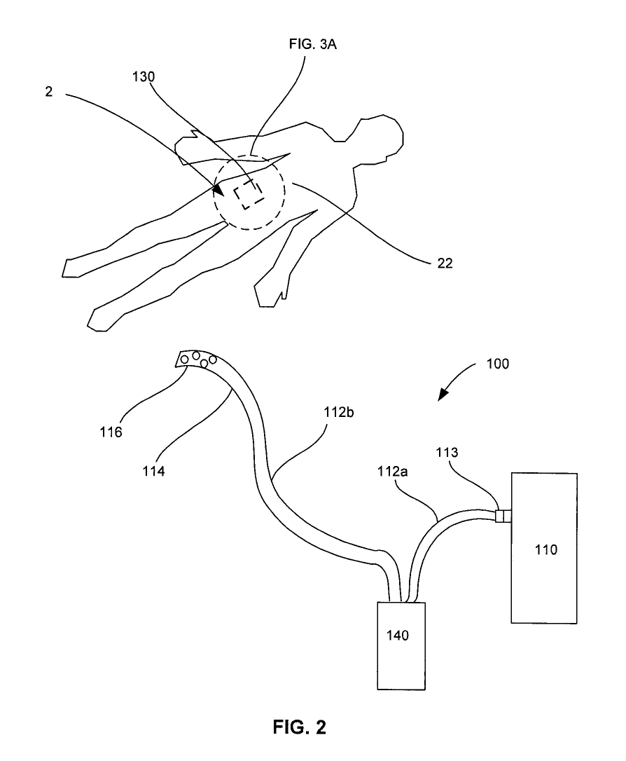

Referring now to FIG. 1, a kit according to one embodiment of the invention includes an implant 130 intended and configured for placement inside the body of the patient. In one example, the implant 130 may be a battery for an MCSD. The battery in this example has an outer casing, visible in FIG. 1, generally in the form of a smoothed edge rectangular solid. In other embodiments, implant 130 may include an implantable electronic or electromechanical device, such as an electronic controller for a device such as an MCSD, an implantable pump of an MCSD, a wireless power receiver, transcutaneous connectors, or any other device designed for implantation inside the human body, and may have different shapes and sizes. In any of the above variants, the implant 130 can include a tissue ingrowth promoter exposed at one or more outer surfaces of the implant 130, as schematically indicated at 132 in FIG. 1. The tissue ingrowth promoter can include a surface with openings such as a mesh or a knitted surface, a porous or roughened surface or another suitable physical construction which facilitates formation of a strong mechanical bond between tissue in contact with the surface and the implant. The ingrowth promoter may also include chemical or biological materials which tend to promote tissue growth, tissue bonding to the surface, or both. An example of a tissue ingrowth promoter for an implant is described in U.S. Provisional App. No. 62/270,156, hereby incorporated by reference herein in its entirety.

The kit also includes a tube 112 structured to access a cavity or cavities in the body where the implant is disposed. The tube 112 includes a first segment 112a and a second segment 112b having a free end 114. The free end 114 of the tube has a plurality of perforations 116. A collection canister 140 is connected between the first segment 112a and the second segment 112b.

In some variants, the second segment 112b of the tube may be a conventional suction catheter, with the distal end of the catheter constituting the free end 114 of the tube. The free end may include features such as a tapered exterior surface to facilitate introduction of the free end into the body of a patient. The first segment 112a may be any conduit which is adapted to resist collapse when the interior of the conduit is subjected to subatmospheric pressure. For example, a braided or thick-walled flexible tube may be used as the first segment. Optionally, the kit can further include a needle and/or a trocar (not shown) for introducing the tube 112. The size and material of the tube 112 are a matter of design choice and may vary depending on the intended application. The first segment 112a of tube 112 has a fitting 113 adapted for connection to a source of suction 110. The suction source 110 may be a portable vacuum pump, a central vacuum system in a hospital or other healthcare facility, or any other device capable of drawing a fluid from tube 112. Canister 140 desirably is arranged so that liquid drawn into the canister from second section 112b will collect in the canister and will not pass into the second section 112a. Canister 140 may be positioned at any location along the tube, but desirably is remote from free end 114. For example, the canister may be disposed at fitting 113. Where the suction source 110 can accept liquids, the canister 140 may be omitted entirely. In these variants, tube 112 may be a unitary tube without separate sections. Conventional control elements can be provided for manually or automatically regulating the suction applied through tube 112. These may include, for example, manually or automatically operated bleed valves for admitting some air into the tube or canister to reduce the amount of suction, and manual or automatic control elements for controlling operation of suction source 110.

The kit discussed above may be pre-assembled and supplied as a unit, for example in a single package. Alternatively, the elements of the kit may be supplied separately and brought together at the point of use.

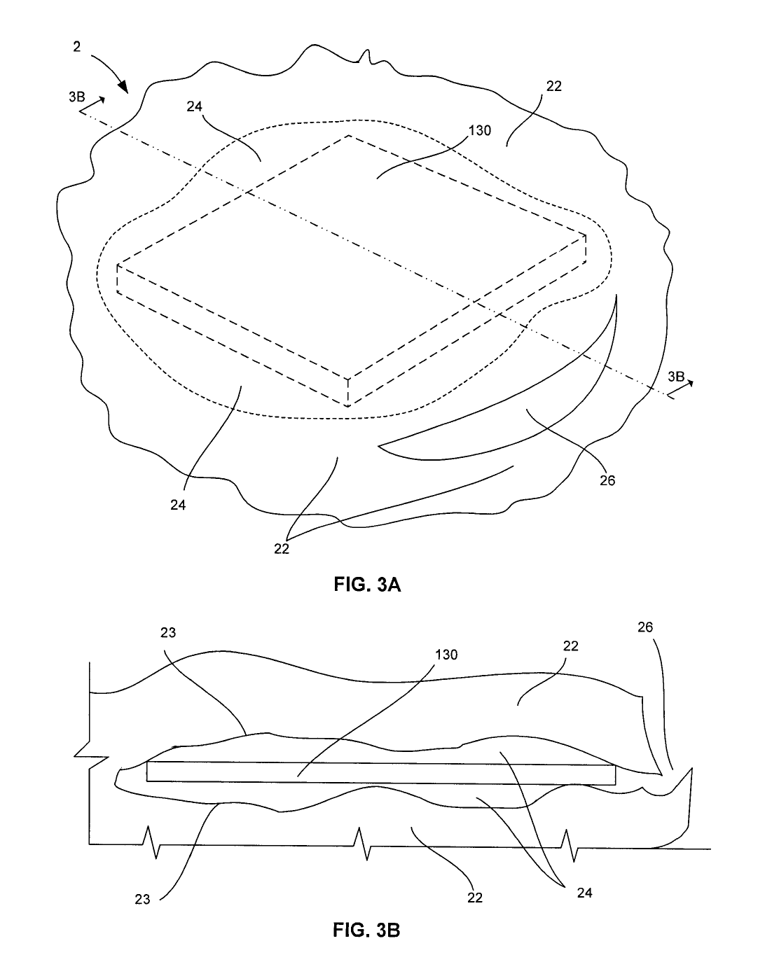

A method according to a further embodiment of the invention can be practiced using the kit 100. The method commences with the placement of an implant 130, such as an implantable electronic or electromechanical device, a battery, a pump, a controller, a wireless power receiver, transcutaneous connectors or any combination thereof, into a cavity 24, as depicted in FIGS. 3A and 3B. In the depicted embodiment, cavity 24 is a surgically created cavity such as a subcutaneous cavity having a surgically-created opening 26 at the skin surface. As best appreciated with reference to FIG. 3B, the tissues 22 of the body bounding the cavity 24 may contact the implant 130 at some locations on its surfaces, whereas at other locations the tissue is out of contact with the implant surfaces. This pattern may vary with the shapes and dimensions of the cavity and implanted device. The space within the cavity which is not filled by the implant may contain air. Moreover, bodily fluids such as blood and interstitial fluid may drain from the tissue into the cavity and collect between the tissue and the implant. At this stage of the process, the tissue and fluids within the cavity typically are at atmospheric pressure.

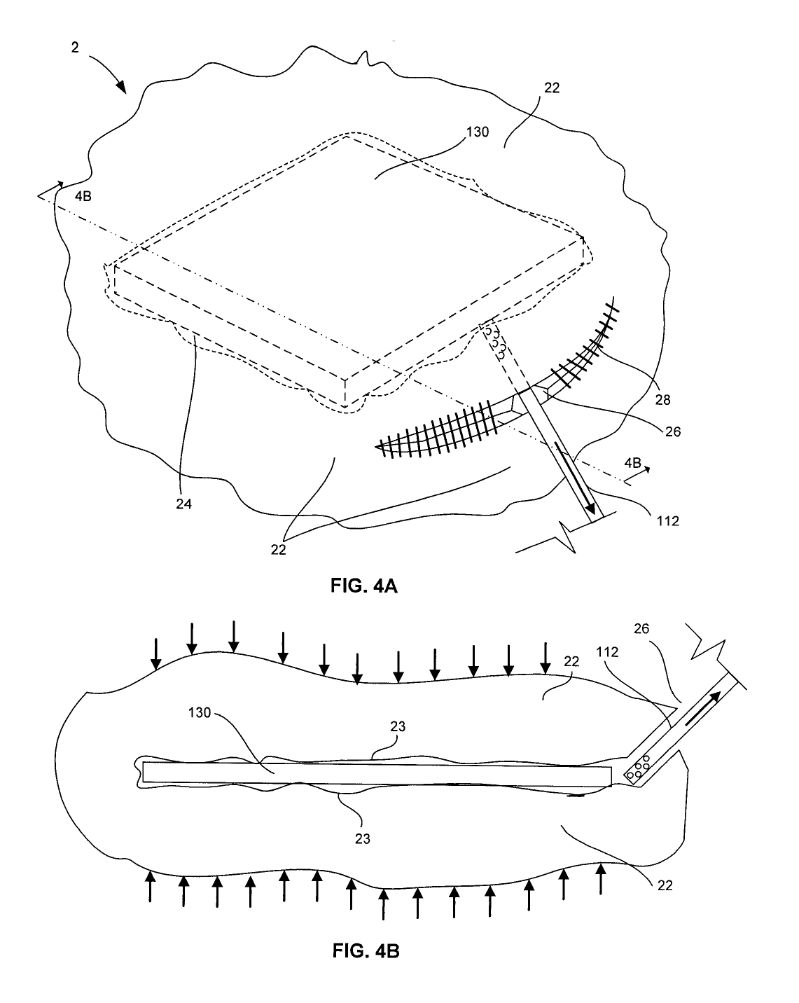

A free end 114 of the tube 112 may be positioned in the body of the patient so that it is inside the cavity 24 surrounding the implant 130. The tube 112 used can be a transcutaneous tube. As shown in FIG. 4, placement of the free end 114 of the tube 112 so that it is inside the cavity 24 is accomplished by advancing the free end of the tube through opening 26. In one configuration, opening 26 is substantially closed by suturing, stapling, application of dressings or other techniques before or after insertion of the tube 112, so that the portion of the tissue immediately adjacent the opening forms at least a partial seal around the tube. This seal may be enhanced by application of dressings (not shown) over the tube. With the free end 114 of the tube in the cavity as shown in FIGS. 4A and 4B, and with the fitting 113 connected to suction source 110, the suction source 110 is actuated to draw fluid from tube 112 and thus create a subatmospheric pressure in the tube 112. Because part of the tube 112 is inside cavity 24, see FIG. 4, fluid in the cavity 24 is evacuated into the tube 112 drawing fluid toward the suction source 110. As fluid is evacuated from the cavity 24, the pressure in the cavity 24 becomes sub-atmospheric. This is in contrast with the surrounding tissue 22, which is typically at atmospheric pressure. The multiple perforations 116 promote uninterrupted evacuation of fluids from the cavity, and prevent application of excessive suction at any one opening. For example, one or two openings at the end of the tube may be blocked by tissue or other obstructions, preventing the evacuation of fluid from those openings. Because additional openings on the tube remain unobstructed, evacuation may continue unimpeded.

The sub-atmospheric pressure caused by the suction through the tube 112 causes tissue walls 23 of the cavity 24 to bear on the surfaces of the implant 130, as visible in comparing the cavity prior to suction, FIG. 3B, and the cavity when subject to suction, FIG. 4B. In FIG. 4B, the arrows around tissue 22 show clamping effect. Put another way, the pressure differential between atmospheric pressure on the surrounding tissues and the subatmospheric pressure within the cavity causes the cavity to diminish in size and close onto the implant 130 so that the tissue walls 23 abut surfaces of the implant 130 to a greater degree. The subatmospheric pressure in the cavity desirably is maintained by keeping tube 112 held in place in the cavity with the suction source 110 in operation running for a period sufficient to allow appreciable healing, which may be about 12 to 72 hours or more. This effect increases the area of contact between the tissue and the surfaces of the implant, and also holds the tissue abutting the implant in intimate contact with the surfaces of the implant under appreciable contact force. This promotes healing of the tissue in intimate contact with the implant. The pressure can be maintained for a duration sufficient so that tissue ingrowth begins. For example, where the surface of the implant allows ingrowth of tissue or otherwise allows the tissue to adhere to the surface, the tissue may bond to the surface of the implant, become secured to the implant, and/or encapsulate the implant. Removal of fluids such as air and liquids through the tube also aids in maintaining intimate contact between the tissue and the implant. Moreover, if the patient moves during the healing process, the implant may tend to move relative to the surrounding tissue under the influence of gravity or other forces. Such movement can damage the relatively weak partially healed tissues and disrupt partially formed bonds between the tissue and the implant. However, the intimate contact and appreciable contact forces between the tissue and the implant tend to prevent relative movement between the implant and the tissue in contact with the implant.

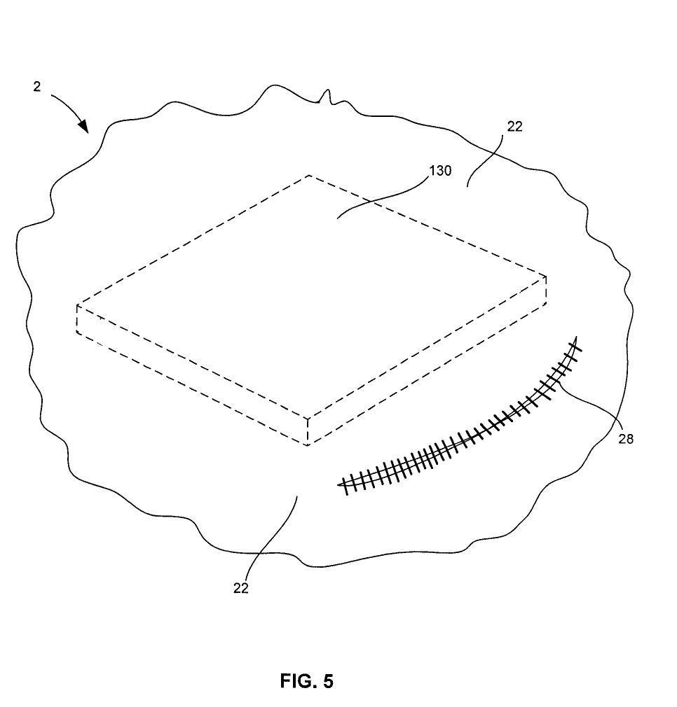

At the end of the evacuation period, the tube 112 is removed and the opening 26 is fully closed, as by suturing 28 shown in FIG. 5. With the implant 130 in its intended position and stabilized, the patient is free to engage in a normal range of physical activities with a minimal risk of the implant tearing from the surrounding tissue and/or becoming disengaged from its implanted location in the tissue 22. Through this procedure, even heavy implants, such as an implantable battery, are stable and secure post-operatively.

The methods and kit described above can be varied in many respects. For example, while the pressure differential between the pressure on the surrounding tissue and the pressure within the cavity desirably is maintained continuously during the healing period, it may be maintained intermittently. Also, the pressure differential can be varied during the healing period as, for example, by varying the subatmospheric pressure within the cavity. In the embodiments discussed above, the cavity is a surgically-created cavity. In other embodiments, the cavity is a naturally occurring cavity or a naturally-occurring cavity which has been enlarged surgically. In the variant shown in FIGS. 3A, 3B and 4A and 4B, the cavity 24 is a single cavity that surrounds the implant 130, and the pressure differential is applied throughout the entire cavity. In other embodiments, the cavity may include separate parts and the pressure differential may be maintained in less than all of these parts. The tube need not be inserted through the same opening used to place the implant. For example, the opening used to place the implant may be closed and the tube may be inserted into the cavity through a separate opening as, for example, an opening formed by a trocar. Where a natural opening of the body communicates with the cavity, the tube 112 can be placed into the patient through a natural opening of the body. In the embodiments discussed above, the opening used to place the implant is partially closed prior to application of the subatmospheric pressure. However, this opening may be left open and closed at a later time, desirably prior to completion of the evacuation procedure. For example, where the tube is used to cause fluid to be evacuated from the cavity for 48 hours, the opening through which the tube passes into the body can be partially closed prior to the end of the 48 hour period. In a variant, closure of the opening can be performed in stages during evacuation of the cavity.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed is:

1. A method of placing an implant in a patient, comprising:
placing an implant consisting of at least one from the group consisting of an electronic device and an electromechanical device into a tissue cavity of the patient; and
evacuating fluid from the cavity and urging tissue surrounding the cavity to abut the implant.

2. The method of claim 1, wherein the cavity is surgically created.

3. The method of claim 2, wherein placing the implant into the cavity includes placing the implant through the surgically created opening in the tissue of the patient.

4. The method of claim 3, further comprising accessing the cavity through the opening to evacuate fluid in the cavity.

5. The method of claim 1, wherein evacuating fluid from the cavity includes lowering the pressure within the cavity to a sub-atmospheric pressure.

6. The method of claim 4, further comprising partially closing the opening prior to evacuating fluid from the cavity.

7. The method of claim 4, further comprising interrupting evacuating fluid by at least a partial closing of the opening in the tissue prior to completing the evacuation of the fluid.

8. The method of claim 1, wherein evacuating fluid is performed for at least 12 hours and a sub-atmospheric pressure is maintained within the cavity for at least 12 hours.

9. The method of claim 1, wherein evacuating fluid is performed until at least a portion of the tissue abuts the implant.

10. The method of claim 9, wherein evacuating fluid is performed until at least one from the group consisting of the tissue surrounding the cavity at least partially heals and the tissue surrounding the cavity secures the tissue with the implant.

11. The method of claim 3, further comprising closing the opening in the tissue and subsequently reopening the closed opening and further comprising at least one from the group consisting of evacuating any fluid not previously removed and evacuating any fluid that has accumulated after closing the opening.

12. The method of claim 1, wherein the implant includes a tissue ingrowth promoter on a surface of the implant, and wherein the tissue ingrowth promoter includes a surface with openings.

13. The method of claim 12, wherein evacuating fluid includes holding tissue of the cavity in abutment with the implant for a duration sufficient for tissue ingrowth into the implant to begin.

14. The method of claim 11, wherein the implant is one from the group consisting of a battery, pump, controller, wireless power receiver, transcutaneous connector, and any combination thereof.

15. The method of claim 1, wherein evacuating fluid includes inserting a transcutaneous tube into an opening in the tissue of the patient and evacuating fluid through the transcutaneous tube.

16. A kit, comprising:
an implant including at least one from the group consisting of an electronic device and electromechanical device sized be disposed in a surgically cavity of a patient;
a tube sized to be received within the cavity; and
a pump connectable to the tube, the pump being configured generate negative pressure that causes suction in the tube.

17. The kit of claim 16, wherein the implant is one of a battery, implantable pump, controller, wireless power receiver, transcutaneous connector, or any combination thereof.

18. The kit of claim 16, wherein the tube includes a suction catheter with a needle configured to penetrate tissue of the patient.

19. The kit of claim 16, further comprising a collection canister, the collection canister being connectable with the tube and configured house fluids suctioned through the tube.

20. A method of placing an implant in a patient, comprising:
surgically creating a tissue cavity in the patient;
placing an implant consisting of a battery into the tissue cavity;
partially closing the tissue cavity;
inserting a transcutaneous tube into the partially closed tissue cavity;
evacuating fluid from the cavity through the transcutaneous tube and urging tissue surrounding the cavity to abut the implant; and
lowering the pressure within the cavity to a sub-atmospheric pressure.
